(19) United States
(12) Patent Application Publication
(10) Pub. No.: US 2023/0205950 A1
(43) Pub. Date: Jun. 29, 2023

(54) AI SIMULATION FOR MICROALGAE FERMENTATION

(71) Applicant: Sophie's BioNutrients Pte. Ltd., Singapore (SG)

(72) Inventors: Yao-Hsin Wang, Singapore (SG); Kirin Tsuei, Taipei (TW)

(73) Assignee: Sophie's BioNutrients Pte. Ltd., Singapore (SG)

(21) Appl. No.: 17/563,103

(22) Filed: Dec. 28, 2021

(51) Int. Cl. G06F 30/27; C12N 1/12; C12M 1/00

(52) U.S. Cl. CPC G06F 30/27; C12M 21/02; C12M 31/10; C12N 1/12; C12N 2500/34

(57) ABSTRACT

A method executed by an engine of a computing device for simulating microalgae fermentation is described. The engine receives an input from a user. The input includes an identification of a microalgae, an identification of a culture media, an identification of an enclosure, and an identification of fermentation conditions for the microalgae when the microalgae is located in the culture media and when the microalgae and the culture media are located in the enclosure. An algorithm of the engine is used to simulate fermentation of the microalgae. A result of the simulation is displayed to the user.

### CROSS-REFERENCE TO RELATED APPLICATIONS SECTION

This application is a Non-Provisional Application that claims benefit from U.S. Provisional Application No. 63132880 filed on Dec. 31, 2020, the entire contents of which are hereby incorporated by reference in their entirety.

### FIELD OF THE EMBODIMENTS

The field of the invention and its embodiments relate to a system and method for the artificial intelligence (AI) simulation of microalgae fermentation.

### BACKGROUND OF THE EMBODIMENTS

Algae are photosynthetic organisms that grow in a range of aquatic habitats, including lakes, pounds, rivers, and oceans. Algae can tolerate a wide range of temperatures, salinities, and pH values, as well as differing light intensities. Additionally, algae may also grow alone or in symbiosis with other organisms. Algae may be broadly classified as Rhodophyta (red algae), Phaeophyta (brown algae), or Chlorophyta (green algae). Algae may be further classified by size, as macroalgae (which are multicellular, large-size algae that are visible with the naked eye) or microalgae (which are microscopic, single cells that may be prokaryotic or eukaryotic).

Currently, there are an estimated 300,000 to 1 million species of microalgae in existence. Microalgae has recently attracted considerable interest due to their extensive applications in the renewable energy field, the biopharmaceutical field, and the nutraceutical field. Specifically, microalgae may be a sustainable and economical source of biofuels, bioactive medicinal products, and food ingredients. Moreover, microalgae also have applications in wastewater treatment and atmospheric CO₂ mitigation. Thus, microalgae produces a wide range of bioproducts, including polysaccharides, lipids, pigments, proteins, vitamins, bioactive compounds, and antioxidants.

Chlorella is a genus of single-celled green algae belonging to the division Chlorophyta. Chlorella I spherical in shape, about 2 to 10 µm in diameter, and is without flagella. It contains the green photosynthetic pigments chlorophyll-a and -b in its chloroplast. Chlorella multiples rapidly, requiring only carbon dioxide, water, sunlight, and a small amount of minerals to reproduce.

Chlorella is a potential food source since it is high in protein and other essential nutrients. For example, when dried, chlorella contains about 45% protein, 20% fat, 20% carbohydrate, 5% fiber, and 10% minerals and vitamins (e.g., vitamin B12, vitamin C, iron, magnesium, zinc, copper, potassium, and/or calcium, etc.). Due to this, chlorella has been labeled as a “superfood” and has garnished significant attention from the vegan community. Further, chlorella has been explored as a potential source of food and energy because its photosynthetic efficiency can, in theory, reach 8%, which exceeds that of other highly efficient crops, such as sugar cane.

Microalgae are feasible sources for bioenergy and biopharmaceuticals in general. With increasing attention being paid to the consumption of healthy nutritional foods, algal protein has moved to the forefront of non-animal protein sources. Since the worlds’ population is projected to escalate to about 9 billion people by 2050, the agricultural sector needs to increase its productivity for maximum yield. Thus, what is needed is an intelligent method to predict and increase this yield.

### SUMMARY OF THE EMBODIMENTS

The present invention and its embodiments provide a system and method for the artificial intelligence (AI) simulation of microalgae fermentation.

A first embodiment of the present invention describes a method executed by an engine of a computing device for simulating microalgae fermentation. In other examples, the engine may alternatively be an application, a software program, a service, and a software platform configured to be executable on the computing device. The method includes numerous process steps, such as: receiving an input from a user. The input may include an identification of a microalgae, an identification of a culture media, an identification of an enclosure, and an identification of fermentation conditions for the microalgae when the microalgae is located in the culture media and when the microalgae and the culture media are located in the enclosure.

In some examples, the microalgae comprise a photoreceptor sensitive to a region of a visible spectrum. Moreover, in examples, the microalgae is of a mixotrophic strain and is adapted for autotrophic growth and heterotrophic growth during a time period. Additionally, the microalgae is a strain selected from the group consisting of: a Botryococcus sudeticus strain, a Botryococcus strain, a Neochloris oleabundans strain, a Neochloris strain, a Chlorella sorokiniana strain, a Chlamydomonas reinhardtii strain, and a Chlamydomonas strain.

The enclosure is a bioreactor (e.g., a fermentation tank) or a photobioreactor that comprises one or more holes. The enclosure further comprises one or more light sources implanted into each of the one or more holes. Each of the one or more light sources produce an irradiance of light in the region of the visible spectrum in a sufficient intensity to transduce the photoreceptor of the microalgae. Moreover, in some examples, each of the one or more light sources includes an artificial light source. In some examples, the artificial light source is a light-emitting diode (LED).

The culture media comprises a carbon source, such as glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, or molasses.

Next, the method may include utilizing an algorithm of the engine to simulate fermentation of the microalgae. The algorithm may include an artificial intelligence (AI) algorithm or a machine learning algorithm. The method may then display a result of the simulation to the user via a display. The result may be displayed as a graph, predictive analytics, and/or visual analytics, among other methods.

Optionally, the method may additionally include: receiving, from the user, a modification of a parameter associated with one of the fermentation conditions to vary the result. The parameter may include: a pH level of the microalgae, a wavelength of irradiance of light onto the microalgae during the fermentation process, a type of light used during the fermentation process, a feedstock for the microalgae, a carbon source of the culture media in which the microalgae is located, a growth temperature for the microalgae, a flow rate of air into the enclosure (e.g., the bioreactor or the photobioreactor) during the fermentation process, a flow rate of air/O₂ mixtures into the enclosure (e.g., the bioreactor or the photobioreactor) during the fermentation process, a length of the fermentation process of the microalgae, a flow rate of noble gases into the enclosure (e.g., the bioreactor or the photobioreactor) during the fermentation process, and/or an incubation time period for the microalgae under the mixotrophic growth condition, among others. The varied result may include: a varied color of the microalgae, a varied aroma of the microalgae, a varied texture of the microalgae, a varied viscosity of the microalgae, and/or a varied nutritional value of the microalgae, among others.

A second embodiment of the present invention describes a system for simulating microalgae fermentation. The system generally includes a network, a computing device, and a server, among other components not explicitly listed herein. The computing device includes, at least, a memory coupled to a processor, a graphical user interface (GUI), and a display. The processor executes an engine such that the engine is configured to: receive, via the GUI, an input from a user. The input comprises an identification of a microalgae, an identification of a culture media, an identification of an enclosure, and an identification of fermentation conditions for the microalgae when the microalgae is located in the culture media and when the microalgae and the culture media are located in the enclosure.

The engine is further configured to: utilize an algorithm to simulate fermentation of the microalgae and display a result of the simulation to the user via the display. Furthermore, the engine is configured to: receive, via the GUI and from the user, a modification of a parameter associated with one of the fermentation conditions to vary the result. The parameter may include: a pH level of the microalgae, a wavelength of irradiance of light onto the microalgae during the fermentation process, a type of light used during the fermentation process, a feedstock for the microalgae, a carbon source of the culture media in which the microalgae is located, a growth temperature for the microalgae, a flow rate of air into the enclosure (e.g., the bioreactor or the photobioreactor) during the fermentation process, a flow rate of air/O₂ mixtures into the enclosure (e.g., the bioreactor or the photobioreactor) during the fermentation process, a length of the fermentation process of the microalgae, a flow rate of noble gases into the enclosure (e.g., the bioreactor or the photobioreactor) during the fermentation process, and/or an incubation time period for the microalgae under the mixotrophic growth condition, among others.

The engine is also configured to: utilize the algorithm to simulate fermentation of the microalgae and display a varied result of the simulation to the user via the display. The varied result may include: a varied color of the microalgae, a varied aroma of the microalgae, a varied texture of the microalgae, a varied viscosity of the microalgae, and/or a varied nutritional value of the microalgae, among others.

The server houses a database and is configured to: store the input, the parameter, the result, and the varied result in the database, among other data.

### BRIEF DESCRIPTION OF THE DRAWINGS

FIG. 1 depicts a schematic diagram of a system for simulating microalgae fermentation, according to at least some embodiments described herein.

FIG. 2 and FIG. 3 depict block diagrams of a system for simulating microalgae fermentation, according to at least some embodiments described herein.

FIG. 4 depicts a block diagram of a computing device used within the system of FIG. 2 and FIG. 3, according to at least some embodiments described herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

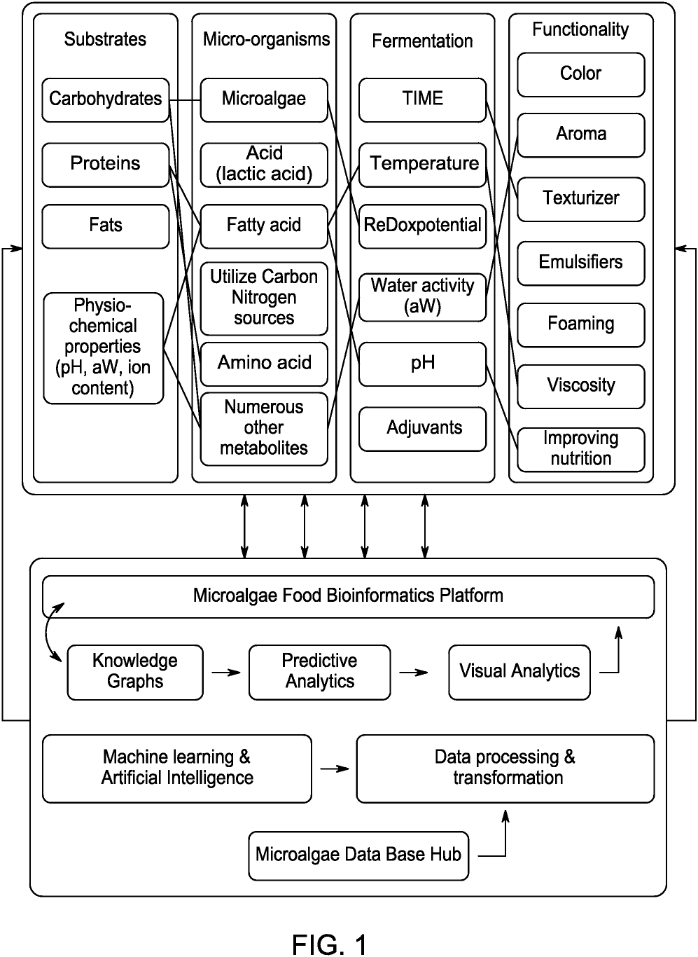

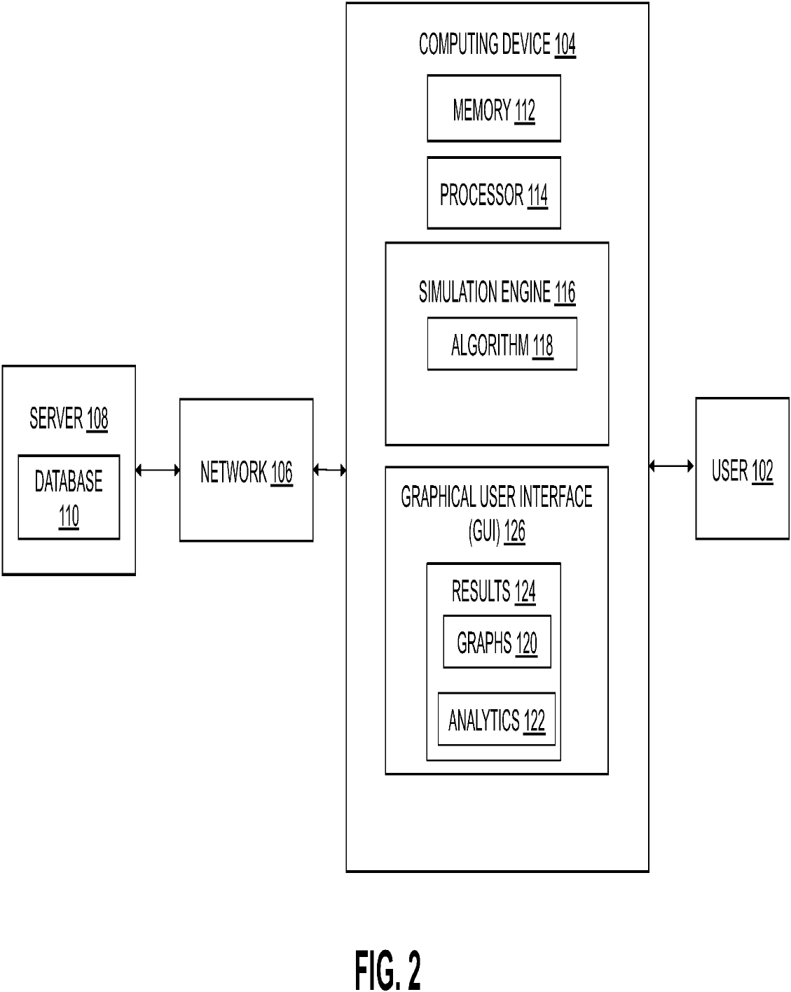

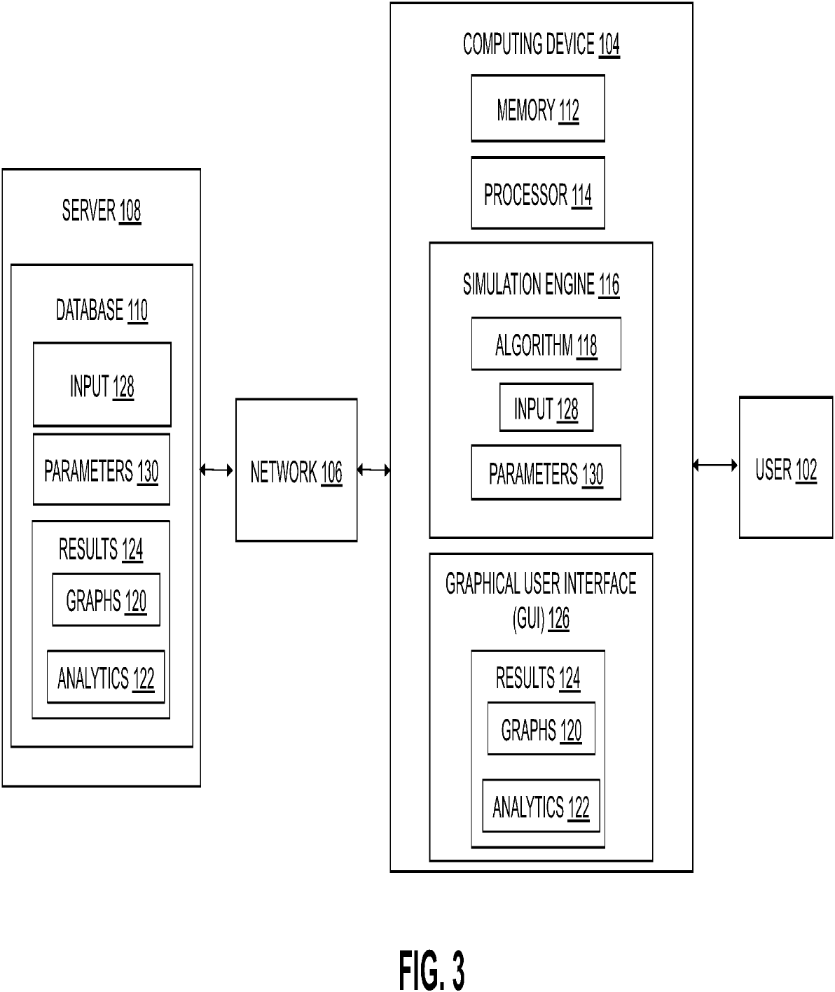

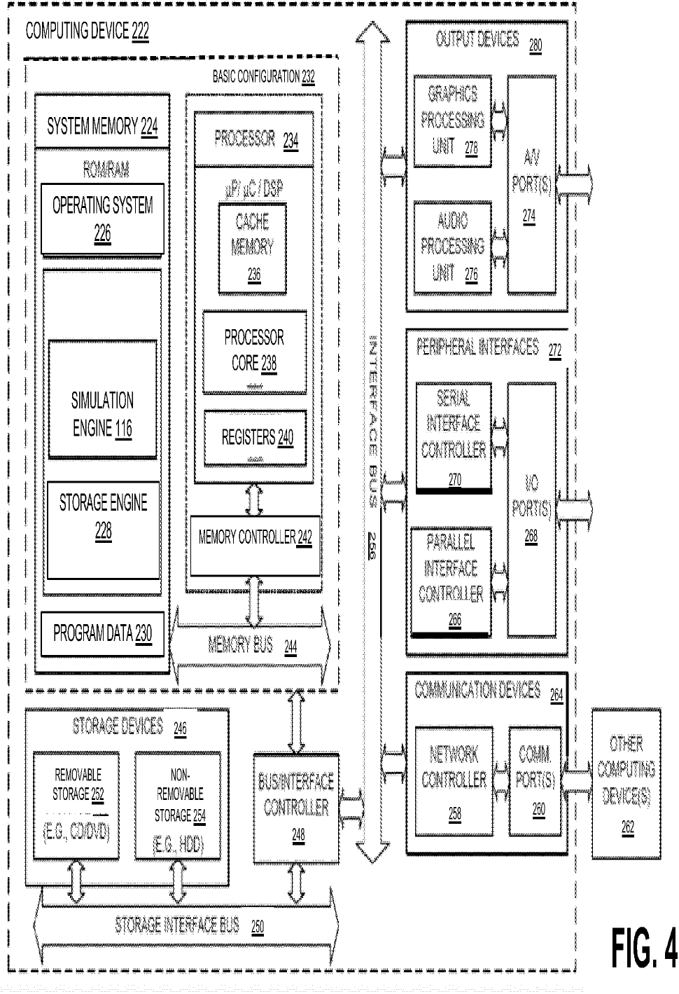

A system and method for simulating microalgae fermentation is described and depicted herein in FIG. 1, FIG. 2, FIG. 3, and FIG. 4. The system of FIG. 2 and FIG. 3 generally includes a network 106, a computing device 104, and a server 108. It should be appreciated that the computing device 104 may be a computer, a laptop computer, a smartphone, and/or a tablet, among other examples not explicitly listed herein. The computing device 104 includes, at least, a memory 112 coupled to a processor 114, a graphical user interface (GUI) 126 and a display.

The processor 114 executes one or more engines or modules, such as a simulation engine 116. In alternative examples, the simulation engine 116 may be an application, a software program, a service, or a software platform configured to be executable on the computing device 104. The simulation engine 116 is configured to perform numerous process steps, such as: receiving, via the GUI 126, an input 128 from a user 102. The input 128 comprises an identification of a microalgae, an identification of a culture media, an identification of an enclosure, and an identification of fermentation conditions for the microalgae when the microalgae is located in the culture media and when the microalgae and the culture media are located in the enclosure.

As defined herein, “fermentation” refers a metabolic process that produces chemical changes in organic substrates through the action of enzymes. In the context of food production, “fermentation” may refer to any process in which the activity of microorganisms brings about a desirable change to a foodstuff or beverage. In microorganisms, fermentation is the primary means of producing adenosine triphosphate (ATP) by the degradation of organic nutrients anaerobically.

As defined herein, a “microalgae” refers to a eukaryotic microbial organism that contains a chloroplast, and optionally, that is capable of performing photosynthesis, or a prokaryotic microbial organism capable of performing photosynthesis. In some examples, the microalgae comprise a photoreceptor sensitive to a region of a visible spectrum. Moreover, in examples, the microalgae is of a mixotrophic strain. The microalgae is adapted for autotrophic growth and heterotrophic growth during a time period. As defined herein, an organism capable of “autotrophic growth” is one that produces complex organic compounds using carbon from simple substances such as carbon dioxide. As defined herein, an organism capable of “heterotrophic growth” is one that cannot produce its own food. As defined herein, an organism capable of “mixotrophic growth” is one that derives nourishment from both autotrophic and heterotrophic mechanisms.

In some examples, the microalgae is of a freshwater strain. In other examples, the microalgae is of a marine strain. For illustrative purposes only, the microalgae is a Botryococcus sudeticus strain, a Botryococcus strain, a Neochloris oleabundans strain, a Chlorella sorokiniana strain, a Neochloris strain, a Chlamydomonas reinhardtii strain, or a Chlamydomonas strain, among other strains not explicitly listed herein.

In examples, the enclosure is a bioreactor (e.g., a fermentation tank) or a photobioreactor and comprises one or more holes. As described herein, a “bioreactor” is an enclosure or partial enclosure, in which cells are cultured, and optionally in suspension. As described herein, a “photobioreactor” refers to a container, at least part of which is at least partially transparent or partially open, thereby allowing light to pass through, in which one or more microalgae cells are cultured.

The enclosure further comprises one or more light sources implanted into each of the one or more holes. Each of the one or more light sources produce an irradiance of light in the region of the visible spectrum in a sufficient intensity to transduce the photoreceptor of the microalgae. Moreover, in some examples, each of the one or more light sources includes an artificial light source. In some examples, the artificial light source is a light-emitting diode (LED).

It should be appreciated that though light sources are described, in some examples, dark fermentation conditions may be used for the microalgae.

The culture media comprises a carbon source, such as glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, or molasses.

The simulation engine 116 is further configured to: utilize one or more algorithms 118 to simulate fermentation of the microalgae and display a result 124 of the simulation to the user 102 via the display. Simulation means and methods may be described, at least, in the following references, which are incorporated by reference in their entirety herein. See, C.E. de Farias Silva, “Simulation Of Microalgal Growth In A Continuous Photobioreactor With Sedimentation And Partial Biomass Recycling,” Braz. J. Chem. Eng., 2016, Vol. 33, No. 4., Pages 773-781; Xiaochen Huang, et al., “Fermentation of Pigment-Extracted Microalgal Residue Using Yeast Cell-Surface Display: Direct High-Density Ethanol Production with Competitive Life Cycle Impacts,” Green Chemistry, 2020, Vol. 22, Pages 153-162; Y. Phan-thien, “BioMASS v 2.0: A New Tool For Bioprocess Simulation,” All Theses, 2011, accessible at: https://tigerprints.clemson.edu/all_theses/1096; Jianqun Lin, et al., “Computer Simulation of Bioprocess,” INTECH, 2017, Chapter 5, Pages 95-115; and Mauricio Ribas, et al., “A Software For Simulation Of Fermentation Processes,” Proc. Int. Soc. Sugar Cane Technol., 2010, Vol. 27, Pages 1-12.

The one or more algorithms 118 may include an artificial intelligence (AI) algorithm and/or a machine learning algorithm. It should be appreciated that the one or more algorithms 118 are not limited to these examples, as these examples have been provided for illustrative purposes only. The result 124 may be displayed as one or more graphs 120 and/or analytics 122 (e.g., predictive analytics, and/or visual analytics), among other methods or means.

Furthermore, the simulation engine 116 is configured to: receive, via the GUI 126 and from the user 102, a modification of a parameter 130 associated with one of the fermentation conditions to vary the result 124. The parameter 130 may include: a pH level of the microalgae, a wavelength of irradiance of light onto the microalgae during the fermentation process, a type of light used during the fermentation process, a feedstock for the microalgae, a carbon source of the culture media in which the microalgae is located, a growth temperature for the microalgae, a flow rate of air into the enclosure (e.g., the bioreactor or the photobioreactor) during the fermentation process, a flow rate of air/O₂ mixtures into the enclosure (e.g., the bioreactor or the photobioreactor) during the fermentation process, a length of the fermentation process of the microalgae, a flow rate of noble gases into the enclosure (e.g., the bioreactor or the photobioreactor) during the fermentation process, and/or an incubation time period for the microalgae under the mixotrophic growth condition, among others.

As defined herein, a “feedstock” refers to what kind of food waste one uses to feed microalgae. Different feed stocks include differing nitrogen and carbon sources.

It should be appreciated that in some examples, the modification of the parameter 130 associated with the one of the fermentation conditions may vary an amino acid combination of the chlorella protein of the microalgae. In other examples, the modification of the parameter 130 associated with the one of the fermentation conditions may include adding a stimulant to the chlorella protein to change the amino acid combination of the chlorella protein. The stimulant is a substrate and may include a spent grain, okara, or molasses, among other examples.

As defined herein, an “amino acid” refers to an organic compound that contains amine (—NH2) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids. About 500 naturally occurring amino acids are known (though only 20 appear in the genetic code) and can be classified in many ways. They can be classified according to the core structural functional groups’ locations as alpha- (α-), beta- (β-), gamma- (γ-) or delta- (δ-) amino acids; other categories relate to polarity, pH level, and side chain group type (aliphatic, acyclic, aromatic, containing hydroxyl or sulfur, etc.).

Amino acids are the basic building blocks of the body and are organic compounds that contain amine (—NH2) and carboxyl (—COOH) functional groups, along with a side chain (R group) specific to each amino acid. In the form of proteins, amino acid residues form the second-largest component (water is the largest) of human muscles and other tissues. Amino acids are extremely versatile and more than 200 different amino acids exist. The most commonly known are the 22 proteinogenic amino acids.

Amino acids prove to be beneficial in numerous fields. For example, L-methionine and L-arginine work together with Glucosamine, Chondroitin, omega-3 and Methylsulfonylmethane (MSM) to prevent and treat arthritis. L-glutamine, L-arginine and L-cysteine may be useful to improve one’s immune system. Branched-chain amino acids (BCAAs) and especially L-leucine are essential for growth, recovery and maintenance of all muscle tissue. L-arginine, L-methionine, L-cysteine, L-lysine, L-glycine and L-proline boost ones natural skin and nail beauty.

L-arginine, L-carnitine and L-cysteine can significantly improve sperm quality and therefore male fertility. L-cysteine, L-glutathione and L-camitine are powerful antioxidants, which protect ones cells from oxidative stress caused by free radicals. L-arginine and Pine bark Extract improve circulation throughout and protect ones body’s arterial walls. Managing L-tryptophan levels can be good for ones sleep. BCAAs, L-glutamine and L-glycine reduce the risk of inflammatory diseases and chronic pain by strengthening ones immune system.

Magnesium, phytoestrogens and L-arginine help manage menopause by reducing hot flushes. L-arginine, L-lysine, zinc and vitamin C improve digestion and protect one from rectal diseases. L-arginine and Ginkgo biloba improve blood circulation, increasing oxygen and nutrient availability within the ear. Moreover, one may face a reduced risk of diabetes with L-arginine and L-carnitine, zinc, magnesium, chromium and omega-3.

It should be appreciated that changing the amino acid combination of the chlorella protein may result in the creation of functional proteins. Proteins are macromolecules consisting of one or more long chains of amino acid residues. Proteins perform a vast array of functions within organisms, including catalyzing metabolic reactions, DNA replication, responding to stimuli, providing structure to cells, and organisms, and transporting molecules from one location to another. Proteins differ from one another primarily in their sequence of amino acids, which is dictated by the nucleotide sequence of their genes, and which usually results in protein folding into a specific three-dimensional structure that determines its activity.

The simulation engine 116 is also configured to: utilize the one or more algorithms 118 to simulate fermentation of the microalgae and display a varied result of the simulation to the user 102 via the display. The varied result may include: a varied color of the microalgae, a varied aroma of the microalgae, a varied texture of the microalgae, a varied viscosity of the microalgae, and/or a varied nutritional value of the microalgae, among others.

It should be appreciated that the modified chlorella protein may be used as a protein flour with different application functions, different nutritional functions, and/or different functional properties based on the modified factor(s) and/or the applied stimulant(s). Such functional properties performed by proteins in food include: solubility, water absorption and binding, viscosity, gelation, cohesion-adhesion, elasticity, emulsification, fat adsorption, flavor binding, and/or foaming, among others. For example, water absorption and binding may be significant in meats, sausages, breads, and cakes, and may be the result of hydrogen-bonding of water and entrapment of water. Additionally, viscosity may be significant for soups and gravies and may result from thickening. Gelation may be important in meats, curds, and cheeses, and may be a result of protein matrix formation and setting. As such, one can use the modified chlorella protein powder that has similar nutritional, functional, and applicational profiles to the animal protein it is trying to replace.

As shown in FIG. 2 and FIG. 3, the server 106 houses a database 110. The database 110 is configured to: store the input 128, the parameter 130, the results 124, the varied results, and other data.

It should be appreciated that the results 124 described herein are not limited to microalgae and proteins formed from microalgae. The described system and method may also be used to make cooking oil, starch, and other nutrients.

Moreover, in some examples, the results 124 and the varied results may include models that the user 102 may learn from to make predictions in terms of microalgae cultivation (e.g., predictive models). As such, the results 124 and the varied results serve as microalgae bioinformatics. As described herein, “bioinformatics” is a subdiscipline of biology and computer science concerned with the acquisition, storage, analysis, and dissemination of biological data, most often DNA and amino acid sequences.

As an illustrative example, when one grows the microalgae from a 10 liter fermentation bioreactor to a 10,000 liter fermentation bioreactor, one is able to collect relevant data for microalgae substrates (carbohydrates, proteins and fats), and microorganisms, such as Extracellular Polymeric Substances (EPS), which consists of amino acids, fatty acids, and numerous metabolites. Furthermore, the microalgae bioinformatics may be used to identify a specific targeted element, like an essential amino acid, and adjust the method of bioprocessing to enhance this targeted element.

In additional examples, the results 124 and the varied results may include knowledge graphs that structure and co-relate aspects of the microalgae bioprocess. Doing so enables one to better understand the microalgae bioprocess. These results 124 and the varied results will help to make well-guided decisions on processing microalgae for optimal yield and will help maximize nutritional values of the microalgae. Furthermore, the results 124 and the varied results allow one to predict the outcome of the process by using predictive analytics and visualizing intricate parts of each element and its association relative to the digital design data.

This system and method described herein allows one to utilize machine learning and/or AI to process data and generate models to detect any inefficiency and improve microalgae production.

### Computing Device

FIG. 4 is a block diagram of a computing device included within the computer system of FIG. 2 and FIG. 3, in accordance with embodiments of the present invention. In some embodiments, the present invention may be a computer system, a method, and/or the computing device 104 (of FIG. 2 and FIG. 3) or the computing device 222 (of FIG. 4).

A basic configuration 232 of a computing device 222 is illustrated in FIG. 4 by those components within the inner dashed line. In the basic configuration 232 of the computing device 222, the computing device 222 includes a processor 234 and a system memory 224. In some examples, the computing device 222 may include one or more processors and the system memory 224. A memory bus 244 is used for communicating between the one or more processors 234 and the system memory 224.

Depending on the desired configuration, the processor 234 may be of any type, including, but not limited to, a microprocessor (µP), a microcontroller (µC), and a digital signal processor (DSP), or any combination thereof. Further, the processor 234 may include one more levels of caching, such as a level cache memory 236, a processor core 238, and registers 240, among other examples. The processor core 238 may include an arithmetic logic unit (ALU), a floating point unit (FPU), and/or a digital signal processing core (DSP Core), or any combination thereof. A memory controller 242 may be used with the processor 234, or, in some implementations, the memory controller 242 may be an internal part of the memory controller 242.

Depending on the desired configuration, the system memory 224 may be of any type, including, but not limited to, volatile memory (such as RAM), and/or non-volatile memory (such as ROM, flash memory, etc.), or any combination thereof. The system memory 224 includes an operating system 226, one or more engines, such as the simulation engine 116, and program data 230. In some embodiments, the simulation engine 116 may be an application, a software program, a service, or a software platform, as described infra. The system memory 224 may also include a storage engine 228 that may store any information disclosed herein.

Moreover, the computing device 222 may have additional features or functionality, and additional interfaces to facilitate communications between the basic configuration 232 and any desired devices and interfaces. For example, a bus/interface controller 248 is used to facilitate communications between the basic configuration 232 and data storage devices 246 via a storage interface bus 250. The data storage devices 246 may be one or more removable storage devices 252, one or more non-removable storage devices 254, or a combination thereof. Examples of the one or more removable storage devices 252 and the one or more non-removable storage devices 254 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 256 facilitates communication from various interface devices (e.g., one or more output devices 280, one or more peripheral interfaces 272, and one or more communication devices 264) to the basic configuration 232 via the bus/interface controller 256. Some of the one or more output devices 280 include a graphics processing unit 278 and an audio processing unit 276, which are configured to communicate to various external devices, such as a display or speakers, via one or more A/V ports 274.

The one or more peripheral interfaces 272 may include a serial interface controller 270 or a parallel interface controller 266, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 268.

Further, the one or more communication devices 264 may include a network controller 258, which is arranged to facilitate communication with one or more other computing devices 262 over a network communication link via one or more communication ports 260. The one or more other computing devices 262 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 224, the one or more removable storage devices 252, and the one or more non-removable storage devices 254 are examples of the computer-readable storage media. The computer-readable storage media is a tangible device that can retain and store instructions (e.g., program code) for use by an instruction execution device (e.g., the computing device 222). Any such, computer storage media is part of the computing device 222.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 234 of a general purpose computer, special purpose computer, or other programmable data processing apparatus (e.g., the computing device 222) to produce a machine, such that the instructions, which execute via the processor 234 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 222), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer/computing device, partly on the user’s computer/computing device, as a stand-alone software package, partly on the user’s computer/computing device and partly on a remote computer/computing device or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

Another embodiment of the invention provides a method that performs the process steps on a subscription, advertising, and/or fee basis.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method executed by an engine of a computing device for simulating microalgae fermentation, the method comprising:
receiving an input from a user, wherein the input comprises an identification of a microalgae, an identification of a culture media, an identification of an enclosure, and an identification of fermentation conditions for the microalgae when the microalgae is located in the culture media and when the microalgae and the culture media are located in the enclosure;
utilizing an algorithm of the engine to simulate fermentation of the microalgae; and
display a result of the simulation to the user via a display.

2. The method of claim 1, wherein the microalgae comprise a photoreceptor sensitive to a region of a visible spectrum.

3. The method of claim 2,
wherein the enclosure comprises one or more holes,
wherein the enclosure comprises a bioreactor or a photobioreactor, and
wherein the bioreactor comprises a fermentation tank.

4. The method of claim 1, wherein the engine is selected from the group consisting of: an application, a software program, a service, and a software platform configured to be executable on the computing device.

5. The method of claim 3,
wherein the enclosure further comprises one or more light sources implanted into each of the one or more holes, and
wherein each of the one or more light sources produce an irradiance of light in the region of the visible spectrum in a sufficient intensity to transduce the photoreceptor of the microalgae.

6. The method of claim 5, wherein each of the one or more light sources includes an artificial light source.

7. The method of claim 6, wherein the artificial light source is a light-emitting diode (LED).

8. The method of claim 1, wherein the culture media comprises a carbon source.

9. The method of claim 8, wherein the carbon source is selected from the group consisting of: glucose, fructose, sucrose, galactose, xylose, mannose, rhainnose, N-acetylglucosamine, glycerol, floridoside, glucuronic acid, corn starch, depolymerized cellulosic material, sugar cane, sugar beet, lactose, milk whey, and molasses.

10. The method of claim 1, wherein the microalgae is of a mixotrophic strain.

11. The method of claim 10, wherein the microalgae is adapted for autotrophic growth and heterotrophic growth during a time period.

12. The method of claim 10, wherein the microalgae is a strain selected from the group consisting of: a Botryococcus sudeticus strain, a Botryococcus strain, a Neochloris oleabundans strain, a Neochloris strain, a Chlorella sorokiniana strain, a Chlamydomonas reinhardtii strain, and a Chlamydomonas strain.

13. The method of claim 1, wherein the algorithm is selected from the group consisting of: an artificial intelligence (AI) algorithm and a machine learning algorithm.

14. The method of claim 1, further comprising:
receiving, from the user, a modification of a parameter associated with one of the fermentation conditions to vary the result.

15. The method of claim 14, wherein the parameter is selected from the group consisting of: a pH level of the microalgae, a wavelength of irradiance of light onto the microalgae during the fermentation process, a type of light used during the fermentation process, a feedstock for the microalgae, a carbon source of a culture media in which the microalgae is located, a growth temperature for the microalgae, a flow rate of air into the enclosure during the fermentation process, a flow rate of air/O₂ mixtures into the enclosure during the fermentation process, a length of the fermentation process of the microalgae, a flow rate of noble gases into the enclosure during the fermentation process, and an incubation time period for the microalgae under a mixotrophic growth condition.

16. The method of claim 14, wherein the varied result is selected from the group consisting of: a varied color of the microalgae, a varied aroma of the microalgae, a varied texture of the microalgae, a varied viscosity of the microalgae, and a varied nutritional value of the microalgae.

17. The method of claim 1, wherein the result is displayed via a graph, predictive analytics, and/or visual analytics.

18. A system for simulating microalgae fermentation, the system comprising:
a network;
a computing device comprising:
a memory coupled to a processor;
a graphical user interface (GUI);
a display; and
the processor executing an engine, the engine being configured to:
receive, via the GUI, an input from a user, wherein the input comprises an identification of a microalgae, an identification of a culture media, an identification of an enclosure, and an identification of fermentation conditions for the microalgae when the microalgae is located in the culture media and when the microalgae and the culture media are located in the enclosure;
utilize an algorithm of the engine to simulate fermentation of the microalgae;
display a result of the simulation to the user via the display;
receive, via the GUI and from the user, a modification of a parameter associated with one of the fermentation conditions to vary the result;
utilize the algorithm to simulate fermentation of the microalgae; and
display a varied result of the simulation to the user via the display; and
a server housing a database, the server being configured to:
store the input, the parameter, the result, and the varied result in the database.

19. The system of claim 18, wherein the parameter is selected from the group consisting of: a pH level of the microalgae, a wavelength of irradiance of light onto the microalgae during the fermentation process, a type of light used during the fermentation process, a feedstock for the microalgae, a carbon source of a culture media in which the microalgae is located, a growth temperature for the microalgae, a flow rate of air into the enclosure during the fermentation process, a flow rate of air/O₂ mixtures into the enclosure during the fermentation process, a length of the fermentation process of the microalgae, a flow rate of noble gases into the enclosure during the fermentation process, and an incubation time period for the microalgae under a mixotrophic growth condition.

20. The system of claim 18, wherein the varied result is selected from the group consisting of: a varied color of the microalgae, a varied aroma of the microalgae, a varied texture of the microalgae, a varied viscosity of the microalgae, and a varied nutritional value of the microalgae.
